(19) United States
(12) Patent Application Publication
(10) Pub. No.: US 2008/0293801 A1
(43) Pub. Date: Nov. 27, 2008

(54) Solid Amorphous Dispersions of An Mtp Inhibitor For Treatment of Obesity

(75) Inventors: Dwayne T. Friesen, Bend, OR (US); Ravi M. Shanker, Groton, CT (US)

Correspondence Address: PFIZER INC., PATENT DEPARTMENT, MS8260-1611, EASTERN POINT ROAD, GROTON, CT, 06340, US

(73) Assignee: PFizer Inc.

(21) Appl. No.: 10/595,732

(22) PCT Filed: Nov. 1, 2004
(86) PCT No.: PCT/IB04/03581

§ 371 Date: May 8, 2006

Related U.S. Application Data
(60) Provisional application No. 60/519,931, filed on Nov. 14, 2003.

(51) Int. Cl. A61K 31/404; A61P 3/04

(52) U.S. Cl. 514/419

(57) ABSTRACT

A composition comprises a solid amorphous dispersion comprising (S)—N-{2-[benzyl(methyl)amino]-2-oxo-1-phenylethyl}-methyl-5-[4′-(trifluoromethyl)[1,1′-biphenyl]-2-carboxamido]-1H-indole-2-carboxamide and a polymer.

### BACKGROUND

The invention relates to a solid amorphous dispersion comprising a Microsomal Triglyceride Transfer Protein inhibitor (MTP inhibitor) for treatment of obesity.

Obesity is a major public health concern because of its increasing prevalence and associated health risks. Obesity and overweight are generally defined by body mass index (BMI), which is correlated with total body fat and estimates the relative risk of disease. BMI is calculated by weight (in kilograms) divided by the square of the person's height (in meters). Overweight is typically defined as a BMI of 25-29.9 kg/m², and obesity is typically defined as a BMI of 30 kg/m² or more. See, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication No. 98-4083 (1998).

The increase in obesity is of concern because of the excessive health risks associated with obesity, including coronary heart disease, strokes, hypertension, type 2 diabetes mellitus, dyslipidemia, sleep apnea, osteoarthritis, gall bladder disease, depression, and certain forms of cancer (e.g., endometrial, breast, prostate, and colon). The negative health consequences of obesity make it the second leading cause of preventable death in the United States and impart a significant economic and psychosocial effect on society. See, McGinnis M, Foege W H, “Actual Causes of Death in the United States,” JAMA, 270, 2207-12 (1993).

Obesity is now recognized as a chronic disease that requires treatment to reduce its associated health risks. Although weight loss is an important treatment outcome, one of the main goals of obesity management is to improve cardiovascular and metabolic values to reduce obesity-related morbidity and mortality. It has been shown that 5-10% loss of body weight can substantially improve metabolic values, such as blood glucose, blood pressure, and lipid concentrations. Hence, it is believed that a 5-10% intentional reduction in body weight may reduce morbidity and mortality.

Currently available prescription drugs for managing obesity generally reduce weight by inducing satiety or decreasing dietary fat absorption. Satiety is achieved by increasing synaptic levels of norepinephrine, serotonin, or both. For example, stimulation of serotonin receptor subtypes 1B, 1D, and 2C and 1- and 2-adrenergic receptors decreases food intake by regulating satiety. See, Bray G A, “The New Era of Drug Treatment. Pharmacologic Treatment of Obesity: Symposium Overview,” Obes Res., 3(suppl 4), 415s-7s (1995). Adrenergic agents (e.g., diethylpropion, benzphetamine, phendimetrazine, mazindol, and phentermine) act by modulating central norepinephrine and dopamine receptors through the promotion of catecholamine release. Older adrenergic weight-loss drugs (e.g., amphetamine, methamphetamine, and phenmetrazine), which strongly engage in dopamine pathways, are no longer recommended because of the risk of their abuse. Fenfluramine and dexfenfluramine, both serotonergic agents used to regulate appetite, are no longer available for use.

Inhibition of MTP provides a unique approach to reduce both fat absorption and food intake. An example of an MTP inhibitor is (S)—N-{2-[benzyl(methyl)amino]-2-oxo-1-phenylethyl}-1-methyl-5-[4′-(trifluoromethyl)[1,1′-biphenyl]-2-carboxamido]-1H-indole-2-carboxamide (referred to herein as “Drug A”). MTP inhibitors cause weight loss by decreasing food intake and inhibiting intestinal fat absorption. However, high variability and limited efficacy have been observed with crystalline Drug A, which has been attributed to its low aqueous solubility.

Although investigations are on-going, there still exists a need for a more effective and safe therapeutic treatment for reducing or preventing weight-gain.

### SUMMARY OF THE INVENTION

A solid amorphous dispersion comprises (S)—N-{2-[benzyl(methyl)amino]-2-oxo-1-phenylethyl}-1-methyl-5-[4′-(trifluoromethyl)[1,1′-biphenyl]-2-carboxamido]-1H-indole-2-carboxamide (Drug A) and a polymer, wherein at least a major portion of Drug A is in amorphous form and wherein Drug A is present in the solid amorphous dispersion in an amount of at least about 40 wt % of the solid amorphous dispersion.

The foregoing and other objectives, features, and advantages of the invention will be more readily understood upon consideration of the following detailed description of the invention.

### DETAILED DESCRIPTION OF THE PREFERRED EMBODIMENTS

Drug A is (S)—N-{2-[benzyl(methyl)amino]-2-oxo-1-phenylethyl}-1-methyl-5-[4′-(trifluoromethyl)[1,1′-biphenyl]-2-carboxamido]-1H-indole-2-carboxamide having Formula (I):

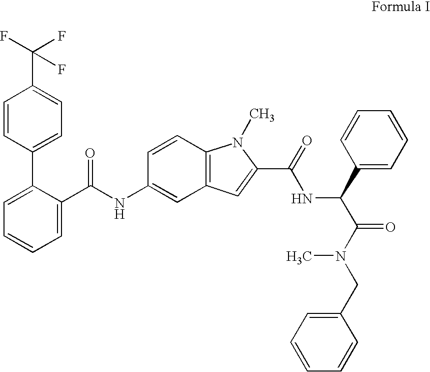

Drug A is disclosed in commonly assigned U.S. provisional patent application Ser. No. 60/301,644 filed Jun. 28, 2001, now U.S. Pat. No. 6,720,351, herein incorporated by reference. Drug A has a molecular weight of about 674.71. Drug A should be understood to include any of its pharmaceutically acceptable forms. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, neutral forms, salt forms and prodrugs.

Drug A is an MTP inhibitor intended for the treatment of obesity. The solubility of the lowest energy crystalline form of Drug A presently known in water is less than 0.6 μg/ml. Drug A is nonionizable, and has a cLog P of about 7.8. These characteristics contribute to its water insoluble nature.

### Concentration-Enhancement

The compositions comprising solid amorphous dispersions of Drug A of the present invention provide concentration enhancement when dosed to an aqueous use environment, meaning that they meet at least one, and preferably both, of the following conditions. The first condition is that the composition increases the maximum drug concentration (MDC) of Drug A in an aqueous use environment relative to a control composition consisting of an equivalent amount of crystalline Drug A in its lowest energy form alone. It is to be understood that the control composition is free from solubilizers or other components that would materially affect the solubility of Drug A in aqueous solution. The control composition is the crystalline form of Drug A alone in its lowest energy, lowest solubility form as presently known. Preferably, the compositions comprising the amorphous dispersions of Drug A provide an MDC of Drug A in an aqueous use environment that is at least 1.25-fold that of the control composition, more preferably at least 2-fold, and most preferably by at least 3-fold that of the control composition.

The second condition is that the compositions comprising solid amorphous dispersions of Drug A increase the dissolution area under the concentration versus time curve (AUC) of Drug A in the aqueous use environment relative to a control composition consisting of an equivalent amount of crystalline Drug A in its lowest energy form alone as presently known. More specifically, in the use environment, the compositions provide an AUC for any 90-minute period of from about 0 to about 270 minutes following introduction to the use environment that is at least 1.25-fold that of the control composition described above. Preferably, the AUC provided by the composition is at least 2-fold, more preferably at least 3-fold that of the control composition.

An “aqueous use environment” can be either the in vivo environment, such as the GI tract of an animal, particularly a human, or the in vitro environment of a test solution, such as phosphate buffered saline (PBS) solution or Model Fasted Duodenal (MFD) solution. An appropriate PBS solution is an aqueous solution comprising 20 mM Na₂HPO₄, 47 mM KH₂PO₄, 87 mM NaCl, and 0.2 mM KCl, adjusted to pH 6.5 with NaOH. An appropriate MFD solution is the same PBS solution wherein there is also present 7.3 mM sodium taurocholic acid and 1.4 mM of 1-palmitoyl-2-oleyl-sn-glycero-3-phosphocholine. The MFD solution may be adjusted to an osmotic pressure of 290 milliosmoles (mOsm) per kg. In particular, a composition formed by the inventive method can be dissolution-tested by adding it to MFD or PBS solution and agitating to promote dissolution. The inventors have found that in vitro dissolution tests are good predictors of in vivo behavior, and thus compositions are within the scope of the invention if they provide concentration-enhancement in either or both in vitro and in vivo use environments. Where the use environment is the GI tract of an animal, dissolved drug concentration may be determined by intubating the patient and periodically sampling the GI tract directly.

An in vitro test to evaluate enhanced Drug A concentration in aqueous solution can be conducted by (1) adding with agitation a sufficient quantity of control composition, typically the crystalline Drug A alone, to the in vitro test medium, such as an MFD or a PBS solution, to achieve equilibrium concentration of Drug A; (2) in a separate test, adding with agitation a sufficient quantity of test composition (e.g., the composition comprising amorphous Drug A) in the same test medium, such that if all Drug A dissolved, the theoretical concentration of Drug A would be at least 2-fold the equilibrium concentration of Drug A, and preferably at least 10-fold; and (3) comparing the measured MDC and/or aqueous AUC of the test composition in the test medium with the equilibrium concentration, and/or with the aqueous AUC of the control composition. In order to quantify the largest enhancements in MDC, the amount of test composition and control composition used should be such that at least a portion of the test composition remains undissolved in the test media at the time of MDC.

The concentration of dissolved Drug A is typically measured as a function of time by sampling the test medium and plotting Drug A concentration in the test medium vs. time so that the MDC can be ascertained. The MDC is taken to be the maximum value of dissolved Drug A measured over the duration of the test. The aqueous AUC is calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, in say less than about 60 minutes, the time interval used to calculate AUC is from time equals zero to time equals 90 minutes. However, if the AUC of a composition over any 90-minute time period described above meets the criterion of this invention, then the composition formed is considered to be within the scope of this invention.

To avoid large drug particulates that would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred dispersions. It should be recognized that this definition of “dissolved drug” encompasses not only monomeric solvated drug molecules but also a wide range of species such as polymer/drug assemblies that have submicron dimensions such as drug aggregates, aggregates of mixtures of polymer and drug, micelles, polymeric micelles, colloidal particles or nanocrystals, polymer/drug complexes, and other such drug-containing species that are present in the filtrate or supernatant in the specified dissolution test.

While it is desired to improve the solubility of Drug A, at least temporarily in the GI tract, it is also desired to limit systemic exposure to Drug A while still maintaining the effectiveness of the drug. Inhibition of fat absorption occurs in the enterocytes of the gut. Systemic exposure of MTP inhibitors (that is, absorption of MTP inhibitors into the blood) is not required nor desired. Therefore, the concentration of dissolved drug in the GI tract is preferably maintained at levels that are high enough to provide efficacy (that is, decrease food intake and fat absorption), but sufficiently low to limit absorption of Drug A into the blood. Thus, in a preferred embodiment, the present invention relates to a composition comprising amorphous Drug A that provides a higher concentration of dissolved Drug A in an aqueous use environment such as the GI tract relative to crystalline drug, such that it is effective at reducing weight of the patient but the concentration of drug dissolved in the GI tract is low enough so that absorption into the blood is limited.

### Solid Amorphous Dispersions

The compositions comprise a solid amorphous dispersion of Drug A and a polymer. By “amorphous” is meant that Drug A is not “crystalline.” By “crystalline” is meant that the drug exhibits long-range order in three dimensions of at least 100 repeat units in each dimension. Thus, the term amorphous is intended to include not only material which has essentially no order, but also material which may have some small degree of order, but the order is in less than three dimensions and/or is only over short distances. Amorphous material may be characterized by techniques known in the art such as powder x-ray diffraction (PXRD) crystallography, solid state NMR, or thermal techniques such as differential scanning calorimetry (DSC). While the compositions of the present invention may contain both amorphous and crystalline Drug A, it is preferred that at least a major portion of Drug A in the composition is in the amorphous form. By “major portion” is meant at least 60 wt %. Preferably, at least 75 wt % of Drug A in the composition is in the amorphous form, and more preferably at least 90 wt % of Drug A is in the amorphous form. Most preferably, the solid amorphous dispersion is substantially free of crystalline Drug A. Amounts of crystalline Drug A may be measured by Powder X-Ray Diffraction (PXRD), Scanning Electron Microscope (SEM) analysis, differential scanning calorimetry (DSC), or any other standard quantitative measurement.

The polymer can exist within the solid amorphous dispersion in relatively pure domains or regions, as a solid solution of polymer homogeneously distributed throughout the amorphous Drug A or any combination of these states or those states that lie intermediate between them. The solid amorphous dispersion is preferably substantially homogeneous so that the amorphous Drug A and polymer are dispersed as homogeneously as possible throughout each other. As used herein, “substantially homogeneous” means that the fraction of Drug A that is present in relatively pure amorphous drug domains or regions within the solid amorphous dispersion is 20 wt % or less. Preferably, the solid amorphous dispersion is almost completely homogeneous, meaning that the fraction of drug present in pure drug domains is 10 wt % or less of the total amount of drug. Solid amorphous dispersions that are at least substantially homogeneous generally are more physically stable and have improved concentration-enhancing properties and, in turn, improved bioavailability, relative to nonhomogeneous dispersions. In a preferred embodiment, the solid amorphous dispersion has at least one glass transition temperature intermediate between that of the drug and the polymer, indicating that at least a portion of the drug and polymer are molecularly dispersed. In a more preferred embodiment, the solid amorphous dispersion has a single glass transition temperature intermediate between that of the drug and the polymer, indicating that the solid amorphous dispersion is completely homogeneous (that is, a solid solution).

The polymer may be selected from the group consisting of hydroxypropyl methyl cellulose acetate succinate (HPMCAS), hydroxypropyl methyl cellulose phthalate (HPMCP), hydroxypropyl methyl cellulose (HPMC), cellulose acetate phthalate (CAP), cellulose acetate trimellitate (CAT), carboxy methyl ethyl cellulose (CMEC), and mixtures thereof.

In a preferred embodiment, the polymer is hydroxypropylmethylcellulose acetate succinate (or “HMPCAS”). As used herein and in the claims, by “HPMCAS” is meant a cellulosic polymer comprising 2-hydroxypropoxy groups (—OCH₂CH(CH₃)OH, hereinafter referred to as hydroxypropoxy groups), methoxy groups (—OCH₃), acetyl groups (—COCH₃), and succinoyl groups (—COCH₂CH₂COOH). Other substituents can be included on the polymer in small amounts, provided they do not materially affect the performance and properties of the HPMCAS.

Generally, the degree of substitution of each substituent group can range from 0.1 to 2.9 as long as the other criteria of the polymer are met. By “degree of substitution” of a substituent or group on HPMCAS is meant the average number of that substituent that is substituted on the saccharide repeat unit on the cellulose chain. The substituent may be attached directly to the saccharide repeat unit by substitution for any of the three hydroxyls on the saccharide repeat unit, or they may be attached through a hydroxypropoxy substituent, the hydroxypropoxy substituent being attached to the saccharide repeat unit by substitution for any of the three hydroxyls on the saccharide repeat unit. For example, if two of the three hydroxyls on the saccharide repeat unit have been substituted with a methoxy group, the degree of substitution of methoxy groups would be 2.0.

HPMCAS is commercially available from Shin-Etsu Chemical (Tokyo, Japan), known by the trade name “AQOAT.” Shin-Etsu manufactures three grades of AQOAT that have different substitution patterns to provide enteric protection at various pH levels. The AS-LF and AS-LG grades (the “F” standing for fine and the “G” standing for granular) provide enteric protection up to a pH of about 5.5. The AS-MF and AS-MG grades provide enteric protection up to a pH of about 6.0, while the AS-HF and AS-HG grades provide enteric protection up to a pH of about 6.8. However, it should be noted that the objective of using HPMCAS in the dispersions of the invention is not to provide enteric protection, but to increase the aqueous concentration of Drug A.

Shin Etsu gives the following specifications for these three grades of AQOAT polymers:

| | Composition of Shin Etsu's AQOAT Polymers (wt %) | | |
|---|---|---|---|
| Substituent | L Grades | M Grades | H Grades |
| Methoxyl Content | 20.0-24.0 | 21.0-25.0 | 22.0-26.0 |
| Hydroxypropoxyl | 5.0-9.0 | 5.0-9.0 | 6.0-10.0 |
| Content | | | |
| Acetyl Content | 5.0-9.0 | 7.0-11.0 | 10.0-14.0 |
| Succinoyl | 14.0-18.0 | 10.0-14.0 | 4.0-8.0 |

A preferred polymer is the H grade of HPMCAS.

Drug A is present in the solid amorphous dispersion in an amount of at least about 40 wt % of the solid amorphous dispersion (or a drug to polymer ratio of at least about 0.66). Drug A may be present in greater amounts, and may be present in the solid amorphous dispersion in an amount of at least about 50 wt % (or a drug to polymer ratio of at least about 1), in an amount of at least about 60 wt % (or a drug to polymer ratio of at least about 1.5), or even in an amount of at least about 75 wt % (or a drug to polymer ratio of at least about 3). In a preferred embodiment, drug A is present in the solid amorphous dispersion in an amount of at least about 85 wt % of the solid amorphous dispersion (or a drug to polymer ratio of at least about 5.7). Dispersions with high drug loadings tend to provide lower dissolved drug concentrations relative to solid amorphous dispersions having lower drug loading. Dispersions having high drug loadings are capable of achieving higher dissolved drug concentration in an aqueous use environment relative to crystalline drug, but also limit systemic exposure relative to dispersions with lower drug loadings. The solid amorphous dispersion may comprise at least about 90 wt %, or even at least about 95 wt % Drug A. Thus, for example, the solid amorphous dispersion may have a drug to polymer ratio of at least about 9, or even at least about 19.

In one embodiment, the solid amorphous dispersion comprises from about 85 wt % to about 98 wt % Drug A, and from about 15 wt % to about 2 wt % polymer. In a preferred embodiment, the solid amorphous dispersion comprises from about 90 wt % to about 97 wt % Drug A, and from about 10 wt % to about 3 wt % polymer. In a more preferred embodiment, the solid amorphous dispersion comprises from about 92 wt % to about 96 wt % Drug A, and from about 8 wt % to about 4 wt % polymer.

### Preparation of Solid Amorphous Dispersions

Solid amorphous dispersions of Drug A may be made according to any conventional process that results in at least a major portion (at least 60%) of Drug A being in the amorphous state. Such processes include mechanical, thermal and solvent processes. Exemplary mechanical processes include milling and extrusion; melt processes including high temperature fusion, solvent-modified fusion and melt-congeal processes; and solvent processes including non-solvent precipitation, spray-coating and spray-drying. Often, processes may form the dispersion by a combination of two or more process types. For example, when an extrusion process is used the extruder may be operated at an elevated temperature such that both mechanical (shear) and thermal means (heat) are used to form the dispersion. Examples of exemplary methods are disclosed in the following U.S. patents, the pertinent disclosures of which are incorporated herein by reference: U.S. Pat. Nos. 5,456,923 and 5,939,099, which describe forming dispersions by extrusion processes; Nos. 5,340,591 and 4,673,564, which describe forming dispersions by milling processes; and Nos. 5,707,646 and 4,894,235, which describe forming dispersions by melt congeal processes.

A preferred method for forming dispersions is “solvent processing,” which consists of dissolution of at least a portion of the drug and at least a portion of the polymer in a common solvent. The term “solvent” is used broadly and includes mixtures of solvents. “Common” here means that the solvent, which can be a mixture of compounds, will dissolve at least a portion of the drug and the polymer. Preferably, the drug and polymer are completely dissolved in the common solvent.

After at least a portion of each of the drug and polymer have been dissolved, the solvent is rapidly removed by evaporation or by mixing with a non-solvent. Exemplary processes are spray-drying, spray-coating (pan-coating, fluidized bed coating, etc.), and precipitation by rapid mixing of the drug and polymer solution with CO₂, hexane, heptane, water of appropriate pH, or some other non-solvent. Preferably, removal of the solvent results in a solid dispersion which is substantially homogeneous. To achieve this end, it is generally desirable to rapidly remove the solvent from the solution such as in a process where the solution is atomized and the drug and polymer rapidly solidify.

The resulting solid amorphous dispersion may be phase separated, meaning the drug and polymer are each in separate domains within the dispersion as described above, or may be homogeneously distributed throughout each other to form a single phase. Preferably, removal of the solvent results in the formation of a substantially homogeneous, solid amorphous dispersion. In such dispersions, Drug A and the polymer are dispersed as homogeneously as possible throughout each other and can be thought of as a solid solution of the polymer dispersed in Drug A, wherein the solid amorphous dispersion is thermodynamically stable, meaning that the concentration of the polymer in Drug A is at or below its equilibrium value, or it may be considered to be a supersaturated solid solution where the polymer concentration in Drug A is above its equilibrium value.

The solvent may be removed by spray-drying. The term “spray-drying” is used conventionally and broadly refers to processes involving breaking up liquid mixtures into small droplets (atomization) and rapidly removing solvent from the mixture in a spray-drying apparatus where there is a strong driving force for evaporation of solvent from the droplets. Spray-drying processes and spray-drying equipment are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atm); or (2) mixing the liquid droplets with a warm drying gas; or (3) both (1) and (2). In addition, at least a portion of the heat required for evaporation of solvent may be provided by heating the spray solution.

Solvents suitable for spray-drying can be any compound in which Drug A and polymer are mutually soluble. Preferably, the solvent is also volatile with a boiling point of 150° C. or less. In addition, the solvent should have relatively low toxicity and be removed from the solid amorphous dispersion to a level that is acceptable according to The International Committee on Harmonization (ICH) guidelines. Removal of solvent to this level may require a subsequent processing step such as tray-drying. Preferred solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, 1,1,1-trichloroethane, and tetrahydrofuran. Mixtures of solvents may also be used.

The amount of Drug A and polymer in the spray solution depends on the solubility of each in the spray solution and the desired ratio of drug to polymer in the resulting solid amorphous dispersion. Preferably, the spray solution comprises at least about 1 wt %, more preferably at least about 3 wt %, and even more preferably at least about 10 wt % dissolved solids.

The solvent-bearing feed can be spray-dried under a wide variety of conditions and yet still yield amorphous drug or solid amorphous dispersions with acceptable properties. For example, various types of nozzles can be used to atomize the spray solution, thereby introducing the spray solution into the spray-dry chamber as a collection of small droplets. Essentially any type of nozzle may be used to spray the solution as long as the droplets that are formed are sufficiently small that they dry sufficiently (due to evaporation of solvent) that they do not stick to or coat the spray-drying chamber wall.

Although the maximum droplet size varies widely as a function of the size, shape and flow pattern within the spray-dryer, generally droplets should be less than about 500 μm in diameter when they exit the nozzle. Examples of types of nozzles that may be used to form the solid amorphous dispersions include the two-fluid nozzle, the fountain-type nozzle, the flat fan-type nozzle, the pressure nozzle and the rotary atomizer. In a preferred embodiment, a pressure nozzle is used, as disclosed in detail in commonly assigned copending U.S. patent application Ser. No. 10/351,568, which claimed priority to U.S. Provisional Application No. 60/353,986, filed Feb. 1, 2002, the disclosure of which is incorporated herein by reference.

The spray solution can be delivered to the spray nozzle or nozzles at a wide range of temperatures and flow rates. Generally, the spray solution temperature can range anywhere from just above the solvent's freezing point to about 20° C. above its ambient pressure boiling point (by pressurizing the solution) and in some cases even higher. Spray solution flow rates to the spray nozzle can vary over a wide range depending on the type of nozzle, spray-dryer size and spray-dry conditions such as the inlet temperature and flow rate of the drying gas. Generally, the energy for evaporation of solvent from the spray solution in a spray-drying process comes primarily from the drying gas.

The drying gas can, in principle, be essentially any gas, but for safety reasons and to minimize undesirable oxidation of Drug A or other materials in the solid amorphous dispersion, an inert gas such as nitrogen, nitrogen-enriched air or argon is utilized. The drying gas is typically introduced into the drying chamber at a temperature between about 600 and about 300° C. and preferably between about 800 and about 240° C.

The large surface-to-volume ratio of the droplets and the large driving force for evaporation of solvent leads to rapid solidification times for the droplets. Solidification times should be less than about 20 seconds, preferably less than about 10 seconds, and more preferably less than 1 second. This rapid solidification is often critical to the particles maintaining a uniform, homogeneous dispersion instead of separating into Drug A-rich and polymer-rich phases. In a preferred embodiment, the height and volume of the spray-dryer are adjusted to provide sufficient time for the droplets to dry prior to impinging on an internal surface of the spray-dryer, as described in detail in commonly assigned, copending U.S. patent application Ser. No. 10/353,746 which claimed priority to U.S. Provisional Application No. 60/354,080, filed Feb. 1, 2002, now U.S. Pat. No. 6,763,607, incorporated herein by reference.

Following solidification, the solid powder typically stays in the spray-drying chamber for about 5 to 60 seconds, further evaporating solvent from the solid powder. The final solvent content of the solid dispersion as it exits the dryer should be low, since this reduces the mobility of Drug A molecules in the solid amorphous dispersion, thereby improving its stability. Generally, the solvent content of the solid amorphous dispersion as it leaves the spray-drying chamber should be less than 10 wt % and preferably less than 2 wt %.

Following formation, the solid amorphous dispersion can be dried to remove residual solvent using suitable drying processes, such as tray drying, vacuum drying, fluid bed drying, microwave drying, belt drying, rotary drying, and other drying processes known in the art. Preferred secondary drying methods include vacuum drying, or tray drying under ambient conditions. To minimize chemical degradation during drying, drying may take place under an inert gas such as nitrogen, or may take place under vacuum.

The solid amorphous dispersion is usually in the form of small particles. The mean size of the particles may be less than 500 μm in diameter, less than 200 μm in diameter, less than 100 μm in diameter or less than 50 μm in diameter. In one embodiment, the particles have a mean diameter ranging from 1 to 100 microns, and preferably from 1 to 50 microns. When the solid amorphous dispersion is formed by spray-drying, the resulting dispersion is in the form of such small particles. When the solid amorphous dispersion is formed by other methods such by melt-congeal or extrusion processes, the resulting dispersion may be sieved, ground, or otherwise processed to yield a plurality of small particles.

For ease of processing, the dried particles may have certain density and size characteristics. In one embodiment, the resulting solid amorphous dispersion particles are formed by spray drying and may have a bulk specific volume of less than or equal to about 4 cc/g, and more preferably less than or equal to about 3.5 cc/g. The particles may have a tapped specific volume of less than or equal to about 3 cc/g, and more preferably less than or equal to about 2 cc/g. The particles have a Hausner ratio (ratio of the bulk specific volume to tapped specific volume) of less than or equal to about 3, and more preferably less than or equal to about 2. The particles may have a Span of less than or equal to 3, and more preferably less than or equal to about 2.5. As used herein, “Span,” is defined as

$$Span = \frac{{D}_{90} - {D}_{10}}{{D}_{50}} ,$$

where D₁₀ is the diameter corresponding to the diameter of particles that make up 10% of the total volume containing particles of equal or smaller diameter, D₅₀ is the diameter corresponding to the diameter of particles that make up 50% of the total volume containing particles of equal or smaller diameter, and D₉₀ is the diameter corresponding to the diameter of particles that make up 90% of the total volume containing particles of equal or smaller diameter.

### Dosage Forms

The compositions may be used in a wide variety of dosage forms for administration of drugs. Exemplary dosage forms are powders or granules that may be taken orally either dry or reconstituted by addition of water or other liquids to form a paste, slurry, suspension or solution; tablets; capsules; multiparticulates; and pills. Various additives may be mixed, ground, or granulated with the compositions of this invention to form a material suitable for the above dosage forms.

The compositions of the present invention may be formulated in various forms such that they are delivered as a suspension of particles in a liquid vehicle. Such suspensions may be formulated as a liquid or paste at the time of manufacture, or they may be formulated as a dry powder with a liquid, typically water, added at a later time but prior to oral administration. Such powders that are constituted into a suspension are often termed sachets or oral powder for constitution (OPC) formulations. Such dosage forms can be formulated and reconstituted via any known procedure. The simplest approach is to formulate the dosage form as a dry powder that is reconstituted by simply adding water and agitating. Alternatively, the dosage form may be formulated as a liquid and a dry powder that are combined and agitated to form the oral suspension. In yet another embodiment, the dosage form can be formulated as two powders that are reconstituted by first adding water to one powder to form a solution to which the second powder is combined with agitation to form the suspension.

In one embodiment, the dosage form is an immediate release tablet. The tablet formulation consists of the solid amorphous dispersion, diluents such as microcrystalline cellulose (Avicel® PH102), and lactose monohydrate (Fast Flo 316®), a disintegrant such as sodium starch glycolate (Explotab®), and a lubricant such as magnesium stearate. An exemplary tablet may be formed by blending about 5 wt % of the solid amorphous dispersion, 59 wt % of microcrystalline cellulose, 32 wt % of lactose monohydrate, and 3 wt % sodium starch glycolate. 0.5 wt % of the lubricant magnesium stearate is then added and the mixture is blended again. The mixture is then granulated with a roller compactor and then milled. An additional 0.5 wt % of the lubricant magnesium stearate is added and the mixture is again blended. The resulting mixture is then placed into a tablet press and compressed.

Other features and embodiments of the invention will become apparent from the following examples which are given for illustration of the invention rather than for limiting its intended scope.

### Example 1

This example formed a solid amorphous dispersion of 95 wt % Drug A with 5 wt % polymer by spray drying. First, a spray solution was formed containing 9.5 wt % Drug A, 0.5 wt % hydroxypropylmethyl cellulose acetate succinate (HPMCAS) (sold under the trade name AQOAT-HG, available from Shin Etsu, Tokyo, Japan), and 90 wt % acetone as follows. The HPMCAS and acetone were combined in a container and mixed for about 2 hours, allowing the HPMCAS to dissolve. The resulting mixture had a slight haze after the entire amount of polymer had been added. Next, Drug A was added directly to this mixture, and the mixture stirred for an additional 4 hours. This mixture was then filtered by passing it through a filter with a screen size of 200 μm to remove any large insoluble material from the mixture, thus forming the spray solution.

The solid amorphous dispersion was then formed using the following procedure. The spray solution was pumped using a high-pressure pump to a spray drier (a Niro type XP Portable Spray-Dryer with a Liquid-Feed Process Vessel (“PSD-1”)), equipped with a pressure nozzle (Spraying Systems Pressure Nozzle and Body) (SK 78-21). The PSD-1 was equipped with a 5-foot 9-inch chamber extension. The chamber extension was added to the spray dryer to increase the vertical length of the dryer. The added length increased the residence time within the dryer, which allowed the product to dry before reaching the angled section of the spray dryer. The spray drier was also equipped with a 316 SS circular diffuser plate with 1/16-inch drilled holes, having a 1% open area. This small open area directed the flow of the drying gas to minimize product recirculation within the spray dryer. The nozzle sat flush with the diffuser plate during operation. The spray solution was delivered to the nozzle at about 163 g/min at a pressure of 100 psig. The pump was followed by a pulsation dampener to minimize pulsation at the nozzle. Drying gas (e.g., nitrogen) was delivered through the diffuser plate at a flow rate of 2100 g/min, and an inlet temperature of 110° C. The evaporated solvent and wet drying gas exited the spray drier at a temperature of 50° C. The spray-dried dispersion formed by this process (344 g) was collected in a cyclone, then post-dried using a Gruenberg single-pass convection tray dryer operating at 50° C. for 24 hours. Following drying, the dispersion was then equilibrated with ambient air and humidity (21° C./45% RH) for 2 hours. The properties of the dispersion after secondary drying were as follows:

TABLE 1

| Bulk Properties (After Secondary Drying) | Tray Dried @ 50° C. |
|---|---|
| Bulk Specific Volume (cc/g) | 2.9 |
| Tapped Specific Volume (cc/g) | 1.9 |
| Hausner Ratio | 1.53 |
| Mean Particle Diameter (μm) | 10 |
| D₁₀, D₅₀, D₉₀ * (μm) | 3, 8, 20 |
| Span (D₉₀-D₁₀)/D₅₀ | 2.1 |
| Residual Acetone | 2.2% |
| (Before Secondary Drying) | |

* 10 vol % of the particles have a diameter that is smaller than D₁₀; 50 vol % of the particles have a diameter that is smaller than D₅₀, and 90 vol % of the particles have a diameter that is smaller than D₉₀.

### Control 1

Control 1 consisted of crystalline Drug A (C1) alone having a melting point of 119° C.

### Concentration Enhancement

### In Vitro Dissolution Tests

In vitro dissolution tests were performed with Example 1 to demonstrate that the solid amorphous dispersion provided concentration-enhancement of Drug A relative to crystalline drug. Samples of Example 1 and Control C1 were added to respective microcentrifuge tubes in duplicate. For these tests, a sufficient amount of material was added so that the maximum theoretical concentration of drug (MTC) would have been 500 μg/mL, if all of the drug had dissolved. The tubes were placed in a 37° C. temperature-controlled chamber, and 1.8 mL model fasted duodenal solution, or “MFDS” was added to each respective tube. The MFDS consisted of 1.8 mL PBS containing 0.5 wt % sodium taurocholic acid and 1-palmitoyl-2-oleyl-sn-glycero-3-phosphocholine (NaTC/POPC, with a 4/1 weight ratio) at pH 6.5 and adjusted to 290 mOsm/kg with NaCl:KCl (20.4:1 wt/wt). The samples were quickly mixed using a vortex mixer for about 60 seconds. The samples were centrifuged at 13,000 G at 37° C. for 1 minute. The resulting supernatant solution was then sampled and diluted 1:6 (by volume) with methanol and then analyzed by high-performance liquid chromatography (HPLC) using a Phenomenex Luna, phenyl-hexyl 5 μm column with a mobile phase consisting of 70:30 (vol:vol) acetonitrile: water at a flow rate of 1 ml/min. Drug concentration was measured using UV absorbance at 241 nm. The contents of each respective tube were mixed on the vortex mixer and allowed to stand undisturbed at 37° C. until the next sample was taken. Samples were collected at 4, 10, 20, 40, and 90 minutes. The results are shown in Table 2.

TABLE 2

| | | Drug A | |
|---|---|---|---|
| | Time | Concentration | AUC |
| Example | (min) | (μg/mL) | (min* μg/mL) |
| 1 | 0 | 0 | 0 |
| | 4 | 7.0 | 14 |
| | 10 | 6.7 | 55 |
| | 20 | 5.4 | 120 |
| | 40 | 4.6 | 220 |
| | 90 | 5.9 | 480 |
| | 1200 | 4.8 | 6,400 |
| C1 | 0 | 0 | 0 |
| | 4 | 0.0* | 0 |
| | 10 | 2.6 | 8 |
| | 20 | 3.1 | 37 |
| | 40 | 2.6 | 93 |
| | 90 | 0.4 | 170 |
| | 1200 | 0.8 | 800 |

*below detection limit

The concentrations of drug obtained in these samples were used to determine the maximum dissolved drug concentration at 90 minutes (MDC₉₀) and the area under the dissolved drug concentration versus time curve (AUC₉₀) during the initial ninety minutes. The results are shown in Table 3.

TABLE 3

| | Drug Conc. in | | | | | AUC₉₀ |
|---|---|---|---|---|---|---|
| | Dispersion | | | MTC | MDC₉₀ | (min* |
| Ex | (active, wt %) | Polymer | Media | (μg/mL) | (μg/mL) | μg/mL) |
| 1 | 95 | HPMCAS | MFDS | 500 | 7.0 | 480 |
| C1 | — | — | MFDS | 500 | 3.1 | 170 |

As can be seen from the data, the solid amorphous dispersion provided concentration-enhancement over that of crystalline drug alone. MDC₉₀ for Example 1 is 2.3-fold that of the crystalline control C1, and AUC₉₀ for Example 1 is 2.8-fold that of the crystalline control C1.

### Example 2

### In Vivo Tests—Dogs

These tests demonstrated that solid amorphous dispersions consisting of 95 wt % Drug A and 5 wt % HPMCAS provided efficacy of Drug A in dogs. Solid amorphous dispersions consisting of 95 wt % Drug A and 5 wt % HPMCAS were prepared as in Example 1 (AQOAT-HG grade of HMPCAS sold by Shin Etsu, Tokyo, Japan).

Healthy, young adult (24 years of age) male and female beagles dogs weighing 15-19 kg at the start of the treatment period were employed as test subjects. The study consisted of two groups of animals containing 3 male and 3 female dogs, each. Each group of six animals was randomly assigned to receive crystalline drug or the solid amorphous dispersion. The test compounds were provided as powders. The dosing suspension, administered by oral gavage, was provided employing a 0.5% methylcellulose/0.1% Tween 80 aqueous solution as the test vehicle. The dosing suspensions were prepared at 0.08 mg/ml activity so that 5 ml was delivered per kg body weight at a dosage of 0.4 mg/kg. Following a seven day baseline acclimation period, a seven day evaluation study was effected. On days 0 to 6 of the study, each dog received the dosing suspension administered as a single dose at Time 0 on each dosing day via a feeding tube. This was followed by a 0.25 mg/kg water rinse to ensure total delivery of dosing solution. Each test animal was permitted ad libitum access to water and IAMS Mini-Chunks® (The lams Company, P.O. Box 14597, Dayton, Ohio) dry food each day during the study and approximately 0.5-1 hours post-dose.

Reduction in food intake was quantitated by weighing individual food bowls each day prior to feeding and at the end of each 24 hour consumption period during the acclimation period and again during the treatment evaluation period. The difference between the weight of the full bowl prior to feeding and the weight of the bowl and amount of food remaining at the end of the 24 hour consumption period represented the reduction in food intake.

Reduction in body weight was quantitated by weighing individual dogs 2 days before beginning dosing (“day-29”) and day 7 of the evaluation study. The difference between the day −2 weight and the day 7 weight represents the reduction in body weight.

Increase in fecal fat percentage was quantitated by collecting total fecal output from individual dogs every 24 hours prior to administration of the dosing suspension on days 0 to 7 and determining the percentage of the wet weight feces that was fat. The difference between the average wet weight fecal fat percentage on days 5 to 7 and the wet weight fecal fat percentage on day 0 represented the increase in fecal fat. Wet weight fecal fat percentage was determined as follows. Each fecal sample was frozen after collection and then thawed overnight at room temperature and then thoroughly mixed to homogeneity after addition of an equal volume of water. An aliquot (about 5 g) was taken from the total sample, transferred to a tared 50-mL centrifuge tube and weighed (to 0.01 g accuracy). Then about 10 g of glass beads and 10 mL of 0.4% amyl alcohol in absolute ethanol were added to each tube, and the tubes were shaken horizontally for 12 minutes at high speed on a flatbed shaker. The samples were acidified with 3 mL of 2 N HCl, and 30 mL of petroleum ether was added. The tubes were shaken as above for 2 minutes and then centrifuged at 1,000 rpm for 5 minutes to separate the phases. A 25-mL aliquot of the petroleum ether layer from each tube was transferred to a pre-weighed crystallizing dish. An additional 25 mL of petroleum ether was added to each tube and the tubes were shaken 1-2 min and centrifuged as above. Again, 25 mL of the petroleum ether layer was transferred to the appropriate crystallizing dish. This step was repeated. The crystallizing dishes were covered with tissue paper and left overnight in a hood to allow for evaporation. The next morning the crystallizing dishes were again weighed to determine the amount of fecal fat collected. The percentage fecal fat recovered from each sample was then calculated.

Reduction in serum cholesterol concentration was quantitated by collecting 3 mL of blood by venipuncture at a time corresponding to 0 hours post-dose on the day prior to dosing (“day−1”) to day 8. The difference between the average serum cholesterol concentration on days −1 to 0 and the serum cholesterol concentration on day 7 represented the decrease in serum cholesterol.

The results showed that the solid amorphous dispersion provided improved efficacy relative to crystalline drug alone, presumably due to higher dissolved drug concentrations in vivo in the GI tract relative to crystalline drug. The solid amorphous dispersion decreased both food intake and body weight. In addition, fecal fat content increased. The solid amorphous dispersion had a 2.1-fold improvement in food intake decrease, a 1.5-fold improvement in body weight decrease, a 1.7-fold improvement in fecal fat increase, and a 1.7-fold improvement in serum cholesterol decrease.

### Examples 3-4

Solid amorphous dispersions of Drug A were made with various ratios of drug to concentration-enhancing polymer and various concentration-enhancing polymers, using a “mini” spray-drying apparatus. Table 4 lists the concentration of drug in each dispersion and the concentration-enhancing polymers used.

TABLE 4

| | Drug Conc. in | |
|---|---|---|
| Example | Dispersion | |
| No. | (active, wt %) | Polymer* |
| 3 | 50 | HPMCAS-MF |
| 4 | 50 | HPMCP |

*Polymer designations: HPMCAS = hydroxypropyl methyl cellulose acetate succinate, HPMCP = hydroxypropyl methyl cellulose phthalate

The following polymers were used to form dispersions. HPMCAS-MF (hydroxypropyl methyl cellulose acetate succinate) was obtained from Shin Etsu (Tokyo, Japan), as AQOAT-MF (“medium, fine”) (the medium designation refers to the relative pH of dissolution, and the fine designation refers to the powder form). HPMCP HP-55 (hydroxypropyl methyl cellulose phthalate) was also obtained from Shin Etsu.

To prepare dispersions using the mini spray drier, Drug A was mixed in acetone together with a polymer to form a spray solution. Each solution was pumped into a “mini” spray-drying apparatus at a rate of 1.3 mL/min via a Cole Parmer 74900 series rate-controlling syringe pump. The drug/polymer solution was atomized through a Spraying Systems Co. two-fluid nozzle, model no. SU1A using a heated stream of nitrogen (70° C.). The spray solution was sprayed into an 11-cm diameter stainless steel chamber. The resulting solid amorphous dispersion was collected on filter paper, dried under vacuum, and stored in a dessicator. The spray solution compositions are shown in Table 5.

TABLE 5

| | Drug | | Polymer | Acetone |
|---|---|---|---|---|
| Example | Mass | | Mass | Mass |
| No. | (mg) | Polymer | (mg) | (g) |
| 3 | 75 | HPMCAS-MF | 75 | 9.8 |
| 4 | 75 | HPMCP | 75 | 10 |

### In Vitro Dissolution Tests

These tests demonstrate that the amorphous dispersions of the invention provide concentration-enhancement of Drug A in vitro. For each test, dispersions were added to microcentrifuge tubes in duplicate. For these tests, a sufficient amount of material was added so that the maximum theoretical concentration (MTC) would have been 500 μg/mL, if all of the drug had dissolved. The tubes were placed in a 37° C. temperature-controlled chamber, and 1.8 mL PBS containing 0.5 wt % sodium taurocholic acid and 1-palmitoyl-2-oleyl-sn-glycero-3-phosphocholine (NaTC/POPC, with a 4/1 weight ratio) at pH 6.5 and 290 mOsm/kg (model fasted duodenal solution, “MFDS”) was added to each respective tube. The samples were quickly mixed using a vortex mixer for about 60 seconds. The samples were centrifuged at 13,000 G at 37° C. for 1 minute. The resulting supernatant solution was then sampled and diluted 1:6 (by volume) with methanol and then analyzed by HPLC as described above. The contents of each respective tube were mixed on the vortex mixer and allowed to stand undisturbed at 37° C. until the next sample was taken. Samples were collected at 4, 10, 20, 40, and 90 minutes. The results are shown in Table 6.

TABLE 6

| | | Drug A | |
|---|---|---|---|
| | Time | Concentration | AUC |
| Example | (min) | (μg/mL) | (min* μg/mL) |
| 3 | 0 | 0 | 0 |
| | 4 | 49 | 100 |
| | 10 | 15 | 300 |
| | 20 | 13 | 400 |
| | 40 | 16 | 700 |
| | 90 | 24 | 1700 |
| 4 | 0 | 0 | 0 |
| | 4 | 54 | 100 |
| | 10 | 76 | 500 |
| | 20 | 121 | 1500 |
| | 40 | 196 | 4700 |
| | 90 | 259 | 16,000 |
| C1 | 0 | 0 | 0 |
| crystalline | 4 | 0 | 0 |
| Drug A in | 10 | 3 | 0 |
| MFDS | 20 | 3 | 0 |
| | 40 | 3 | 100 |
| | 90 | 0 | 200 |

The concentrations of drug obtained in these samples were used to determine the MDC₉₀ and the AUC₉₀ during the initial ninety minutes. The results are shown in Table 7.

TABLE 7

| | Drug Conc. in | | | | | AUC₉₀ |
|---|---|---|---|---|---|---|
| | Dispersion | | | MTC | MDC₉₀ | (min* |
| Ex | (wt % A) | Polymer | Media | (μg/mL) | (μg/mL) | μg/mL) |
| 3 | 50 | HPMCAS- | MFDS | 500 | 49 | 1700 |
| | | MF | | | | |
| 4 | 50 | HPMCP | MFDS | 500 | 259 | 16,000 |
| C1 | — | — | MFDS | 500 | 3 | 200 |

As can be seen from the data, the dispersions of the invention provided concentration-enhancement over that of crystalline drug alone.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

What is claimed is:

1. A solid amorphous dispersion comprising a compound having Formula (I) 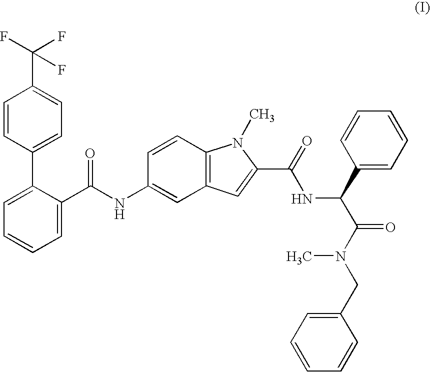
and a polymer, wherein said compound is present in an amount of at least about 40 wt % of said solid amorphous dispersion.

2. The solid amorphous dispersion of claim 1 wherein said compound is present in an amount of at least about 50 wt % of said dispersion.

3. The solid amorphous dispersion of claim 1 wherein said compound is present in an amount of at least about 75 wt % of said dispersion.

4. The solid amorphous dispersion of claim 1 wherein said compound is present in an amount of at least about 85 wt % of said dispersion.

5. The solid amorphous dispersion of claim 1 wherein said compound is present in an amount of at least about 90 wt % of said dispersion.

6. The solid amorphous dispersion of claim 1 wherein said compound is present in an amount of about 95 wt % of said dispersion.

7. The solid amorphous dispersion of claim 1 wherein said compound is present in an amount of from about 85 wt % to about 98 wt % of said dispersion.

8. The solid amorphous dispersion of claim 1 wherein said compound is present in an amount of from about 90 wt % to about 97 wt % of said dispersion.

9. The solid amorphous dispersion of claim 1 wherein said polymer is selected from the group consisting of hydroxypropyl methyl cellulose acetate succinate (HPMCAS), hydroxypropyl methyl cellulose phthalate (HPMCP), hydroxypropyl methyl cellulose (HPMC), cellulose acetate phthalate (CAP), cellulose acetate trimellitate (CAT), and carboxy methyl ethyl cellulose (CMEC) and mixtures thereof.

10. The solid amorphous dispersion of claim 1 wherein said polymer is hydroxypropylmethyl cellulose acetate succinate.

11. The solid amorphous dispersion of claim 8 wherein said polymer is the H grade of said hydroxypropylmethyl cellulose acetate succinate.

12. The solid amorphous dispersion of claim 1 wherein said solid amorphous dispersion provides a maximum concentration of said compound in an aqueous use environment that is at least 1.25-fold that of a control composition consisting essentially of an equivalent quantity of said compound in crystalline form.

13. The solid amorphous dispersion of claim 1 wherein said composition provides in an aqueous use environment an area under the concentration versus time curve for any period of at least 90 minutes between the time of introduction into the use environment and about 270 minutes following introduction to the use environment that is at least about 1.25-fold that of a control composition consisting essentially of an equivalent quantity of said compound in crystalline form.

14. The solid amorphous dispersion of claim 1 wherein said solid amorphous dispersion has a mean particle diameter of less than about 100 microns.

15. The solid amorphous dispersion of claim 1 incorporated into a tablet.

16. A process for forming a solid amorphous dispersion comprising:
(a) dissolving a compound and a polymer in a solvent to form a spray solution;
(b) rapidly evaporating said solvent from said spray solution to form said solid amorphous dispersion;
wherein said compound has Formula I [chemical structure]
